(19) United States
(12) Patent Application Publication
(10) Pub. No.: US 2013/0281506 A1
(43) Pub. Date: Oct. 24, 2013

(54) COMBINATION OF A NON-STEROIDAL ANTI-INFLAMMATORY DRUG WITH AN ANTI-HISTAMINIC DRUG INTENDED FOR OPHTHALMIC USE

(71) Applicant: ALLERGAN, INC., Irvine, CA (US)

(72) Inventor: Kothanda Raman Thyaga Rajan, Bangalore (IN)

(73) Assignee: Allergan, Inc., Irvine, CA (US)

(21) Appl. No.: 13/866,313

(22) Filed: Apr. 19, 2013

Related U.S. Application Data
(60) Provisional application No. 61/635,506, filed on Apr. 19, 2012.

(51) Int. Cl. A61K 31/407; A61K 31/335

(52) U.S. Cl. CPC A61K 31/407; A61K 31/335
USPC 514/413

(57) ABSTRACT

The present invention is directed to a stable formulation of a combination of ketorolac (non-steroidal anti-inflammatory drug) with olopatadine (anti-histaminic drug) intended for ophthalmic use. This pharmaceutical composition is used for treatment of ophthalmic diseases and conditions, particularly seasonal ocular surface allergy.

### CROSS REFERENCE TO RELATED APPLICATION

This application claims the benefit of U.S. Provisional Patent Application Ser. No. 61/635,506, filed Apr. 19, 2012, the disclosure of which is hereby incorporated in its entirety herein by reference.

### FIELD OF THE INVENTION

The present invention is directed to a stable formulation of a combination of ketorolac (non-steroidal anti-inflammatory drug) with olopatadine (anti-histaminic drug) intended for ophthalmic use. This pharmaceutical composition is used for treatment of ophthalmic diseases and conditions, in particular ocular surface allergy.

### BACKGROUND OF THE INVENTION

Ocular surface allergy, in particular allergic conjunctivitis, is an immunologically mediated inflammatory condition. Most common symptoms include: pronounced bilateral red, swollen, and itchy eyes, a milky conjunctival appearance, a stringy, ropy, or watery discharge, and papillary hypertrophy of the tarsal conjunctiva in severe cases.

The pathogenesis of ocular surface allergy, allergic conjunctivitis, is complex and involves multiple mechanisms that result in mast cell degranulation and the release of allergic mediators such as histamine, prostaglandins, and other products of the arachidonic acid metabolism. These mediators produce effects on the eye such as conjunctival vasodilation, increased vascular permeability, leukocyte chemotaxis, and surface destruction (Raizman M B, “Results of a survey of patients with ocular allergy treated with topical ketorolac tromethamine” Clinical Therapeutics, 1995, 17(5), 882-890).

### SUMMARY OF INVENTION

The present invention is directed to a combination of a non-steroidal anti-inflammatory drug (NSAID) with an antihistamine intended for ophthalmic use.

The non-steroidal anti-inflammatory drug (NSAID) is selected from, but not limited to: indomethacin, diclofenac, flurbiprofen, ketorolac, suprofen, bromfenac, nepafenac. The ophthalmic antihistamine is selected from, but not limited to: olopatadine, ketotifen, epinastine, alcaftadine, azelastine, cetitizine, axelastine, emedastine, bepotasine, chlorpheneramine, clemastine, diphenhydramine, loratadine, pheniramine. The present invention is directed to a unique and efficacious combination of ketorolac with olopatadine intended for an ophthalmic use.

The pharmacological approach to ocular surface allergy is complex and there is great variability in treating patients with ocular allergies. The treatment of allergic conjunctivitis depends on the severity of the patient's signs and symptoms. There are a number of topical ophthalmic products that are used to treat ophthalmic allergies e.g. antihistamines, mast cell stabilizers, non-steroidal anti-inflammatory drugs, steroids. There is a strong need for improved ophthalmic compositions of mast cell stabilizers or antihistamines with non-steroidal anti-inflammatory agents used in methods of treatment or preventing of ophthalmic symptoms of ocular surface allergy in particular allergic conjunctivitis.

The use of antihistamines with mast cell stabilizer may inactivate the immediate cascade of histamine associated responses during the early phase of the hypersensitivity reaction; however, they have no effect on the newly formed mediators such as prostaglandins, except PGD₂, which in combination with histamine was shown to be additive in producing ocular itching, they also continue to produce ocular allergy symptoms for several hours after the allergen exposure. Prostaglandins E₁ and E₂ have been found to produce vasodilation and erythema, potentiate edema, and sensitize nerve endings to agents that induce pain. While the levels of histamine dissipate because of metabolism in the ocular tissue, the administration of ketorolac tromethamine inhibits the further synthesis of prostaglandins. Thereby, it is expected that a combination of mast cell stabilizer with antihistamine, e.g. olopatadine with the non-steroidal anti-inflammatory drug like ketorolac, will help in disrupting the complex chain of events that make up the allergic conjunctivitis at various levels and thereby produces a synergistic effect in reducing the signs and symptoms of allergic conjunctivitis.

The combination of olopatadine, a dual acting mast cell stabilizer or antihistaminic agent with ketorolac, a highly potent non steroidal anti-inflammatory drug, was explored. Olopatadine, a dihydrodibenzooxepine, is a dual acting mast cell stabilizer or an antihistaminic agent. It is a selective H¹ receptor antagonist and an inhibitor of mast-cell degranulation, preventing the release of histamine and other mediators of allergic immune responses. However, olopatadine has no action on prostaglandins except PGD₂, which are “newly formed” mediators produced by the arachidonic acid pathway, and are involved in initiating the late-phase inflammatory reaction. Ketorolac, a dihydropyrrolizine, is a highly potent non steroidal anti-inflammatory drug (NSAID). Its primary mechanism of action is on the arachidonic acid cascade, by binding to the cyclooxygenase and blocking the production of prostaglandins. Ketorolac tromethamine is the only available topical NSAID preparation, indicated for the relief of ocular itching associated with seasonal allergic conjunctivitis, the most prevalent form of ocular allergy. Currently these drugs are individually approved and commercially available on the market for the treatment for seasonal allergic conjunctivitis.

Since allergic conjunctivitis was associated with inflammation, there is a need to co-prescribe both classes of the drugs individually. The current invention provides a pharmaceutically stable formulation of both classes of drugs combined in a single formulation. The idea was to act upon the cascade of events, which result during the allergic conjunctivitis, producing a synergistic effect in reducing the signs and symptoms of ocular surface allergy or allergic conjunctivitis.

The concentration of the mast cell stabilizer or antihistamine agent and of the non-steroidal anti-inflammatory drug like agent, contained in the composition of the present invention is based on the type of condition being treated. The concentration is sufficient to reduce the signs and symptoms of ocular surface allergy or allergic conjunctivitis in the targeted ophthalmic tissues following topical application of the compositions to those tissues. Such an amount is referred to as “a therapeutically effective amount”.

The composition of the present invention contains olopatadine hydrochloride in ranges of about 0.01% w/v to about 0.15% w/v, about 0.05% w/v to about 0.15% w/v, about 0.1% w/v to about 0.1% w/v, but most preferable, the concentration of olopatadine hydrochloride is about 0.1% w/v. The composition of the present invention also contains ketorolac tromethamine in ranges of about 0.1% w/v to about 0.5% w/v, about 0.05% w/v to about 0.45% w/v, about 0.1% w/v to about 0.45% w/v, about 0.1% w/v to about 0.4% w/v, but most preferable the concentration of ketorolac tromethamine is about 0.4% w/v. The most preferable concentration of ketorolac tromethamine is 0.4% w/v in combination with olopatadine hydrochloride 0.1% w/v.

In one aspect the invention provides a combination eye drop of olopatadine hydrochloride 0.1% (w/v) with ketorolac tromethamine 0.4% (w/v).

In another aspect the invention provides a combination eye drop of olopatadine hydrochloride 0.1% (w/v) with ketorolac tromethamine 0.4% (w/v) and benzalkonium chloride 0.005% (w/v).

In another aspect the invention provides a topical ophthalmic composition with a pH between 6.6 to 7.1. In another aspect the invention provides a topical ophthalmic composition with a pH between 6.6 to 6.9. In another aspect the invention provides a topical ophthalmic composition with a pH of 6.8.

In another aspect the invention provides a topical ophthalmic preparation comprising ketorolac tromethamine is 0.4% w/v, olopatadine hydrochloride 0.1% w/v, a pH of 6.8, benzalkonium chloride is 0.005% w/v, sodium dihydrogen dihydrate phosphate, disodium hydrogen phosphate anhydrous, sodium hydroxide pellets and water.

In another aspect the invention provides a topical ophthalmic preparation consisting of ketorolac tromethamine is 0.4% w/v, olopatadine hydrochloride 0.1% w/v, a pH of 6.8, benzalkonium chloride is 0.005% w/v, sodium dihydrogen dihydrate phosphate, disodium hydrogen phosphate anhydrous, sodium hydroxide pellets and water.

In another aspect the invention provides a topical ophthalmic preparation comprising a combination of a mast cell stabilizer or antihistamine agent with a non-steroidal anti-inflammatory drug like agent.

In another aspect the invention provides an ophthalmic composition used for the treatment of ophthalmic diseases and conditions, particularly seasonal allergic conjunctivitis.

In another aspect the invention provides an ophthalmic composition used for the treatment of ocular surface allergy.

In another aspect the invention provides an ophthalmic composition comprising a combination of a mast cell stabilizer or antihistamine agent with a non-steroidal anti-inflammatory drug like agent, used for the treatment of ophthalmic diseases and conditions, particularly seasonal allergic conjunctivitis.

In another aspect the invention provides an ophthalmic composition comprising a combination of a mast cell stabilizer or antihistamine agent with a non-steroidal anti-inflammatory drug like agent, used for the treatment of ocular surface allergy.

Other examples of ophthalmic conditions which may be treated with the compositions of the present invention include infective conditions associated with inflammation and where the use of NSAID and the use of anti-histamine are acceptable. Such conditions may include, but are not limited to conjunctivitis, keratitis, blepharitis, dacyrocystitis, hordeolum, corneal ulcers, anterior blepharitis, posterior blepharitis, meibomian gland dysfunction, dry eye disease (keratocojunctivitis sicca), ocular pain and inflammation post-ocular surgery, bacterial conjunctivitis, anterior uveitis, post-surgical inflammation, inflammatory conditions of the palpebral and bulbar conjunctiva, cornea, and anterior segment of the globe, such as allergic conjunctivitis, ocular surface allergy, ocular rosacea, superficial punctate keratitis, herpes zoster keratitis, iritis, cyclitis, selected infective conjunctivitis, corneal injury from chemical radiation, thermal burns, penetration of foreign bodies, allergy, itchy eyes, swelling caused by seasonal allergies, eye pain and sensitivity to light following certain eye surgeries, eye swelling following cataract surgery, eye symptoms of hay fever and combinations thereof.

In another aspect the invention provides an ophthalmic composition comprising a combination of a mast cell stabilizer or antihistamine agent with a non-steroidal anti-inflammatory drug like agent in pharmaceutically acceptable excipients and vehicles with an acceptable pH and viscosity.

In another aspect the invention provides a clear, sterile, stable aqueous composition of a combination of a mast cell stabilizer or antihistamine agent with a non-steroidal anti-inflammatory drug like agent, which when administered topically to the eye, does not cause any irritation or discomfort to the eye.

In another aspect the invention provides a combination of a mast cell stabilizer or antihistamine agent with a non-steroidal anti-inflammatory drug like agent, having more patient compliance and acceptability.

1 ml of the ophthalmic composition of the present invention contains:

Olopatadine hydrochloride USP, equivalent to olopatadine 1.0 mg;

Ketorolac tromethamine IP 4.0 mg;

Benzalkonium chloride IP 0.05 mg;

Purified water q.s.

The composition of the current invention is administered to the affected ophthalmic tissues by topically applying one to four drops of the sterile composition one to four times per day per eye, or more as indicated.

Benzalkonium chloride is used as the preferred preservative in the present invention. However, it is possible that other preservatives commonly used in ophthalmic solutions be utilized such as Purite® (chlorine dioxide), polixetonium, polyhexamethylene biquanide, polyquad and sodium perborate.

In a further embodiment the present invention includes an ophthalmic composition comprising sodium dihydrogen dihydrate phosphate, disodium hydrogen phosphate anhydrous, sodium hydroxide (pellets).

In one embodiment the present invention includes a topical ophthalmic composition for treating or preventing ophthalmic seasonal allergic conjunctivitis in a human patient, wherein the ophthalmic composition contains a combination of olopatadine hydrochloride with ketorolac tromethamine.

In another embodiment the present invention includes a topical ophthalmic composition for treating or preventing ocular surface allergy in a human patient, wherein the ophthalmic composition contains a combination of olopatadine hydrochloride with ketorolac tromethamine.

In a further embodiment the present invention includes an ophthalmic composition, for treating or preventing seasonal allergic conjunctivitis in a human patient, wherein the ophthalmic composition comprises a combination of a non-steroidal anti-inflammatory drug with an antihistamine intended for an ophthalmic use.

In a further embodiment the present invention includes an ophthalmic composition, for treating or preventing ocular surface allergy in a human patient, wherein the ophthalmic composition comprises a combination of a non-steroidal anti-inflammatory drug with an antihistamine intended for an ophthalmic use.

In a further embodiment the present invention includes an ophthalmic composition, for treating or preventing seasonal allergic conjunctivitis in a human patient, wherein the non-steroidal anti-inflammatory drug is selected from: indomethacin, diclofenac, flurbiprofen, ketorolac, suprofen, bromfenac and nepafenac.

In a further embodiment the present invention includes an ophthalmic composition, for treating or preventing ocular surface allergy in a human patient, wherein the non-steroidal anti-inflammatory drug is selected from: indomethacin, diclofenac, flurbiprofen, ketorolac, suprofen, bromfenac and nepafenac.

In a further embodiment the present invention includes an ophthalmic composition, for treating or preventing seasonal allergic conjunctivitis in a human patient, wherein the antihistamine is selected from: olopatadine, ketotifen, epinastine, alcaftadine, azelastine, cetitizine, axelastine, emedastine, bepotasine, chlorpheneramine, clemastine, diphenhydramine, loratadine and pheniramine.

In a further embodiment the present invention includes an ophthalmic composition, for treating or preventing ocular surface allergy in a human patient, wherein the antihistamine is selected from: olopatadine, ketotifen, epinastine, alcaftadine, azelastine, cetitizine, axelastine, emedastine, bepotasine, chlorpheneramine, clemastine, diphenhydramine, loratadine and pheniramine.

In a further embodiment the present invention includes an ophthalmic composition, wherein the composition is useful in preventing signs and symptoms of allergic conjunctivitis.

In a further embodiment the present invention includes an ophthalmic composition, wherein the composition is useful in preventing signs and symptoms of ocular surface allergy.

In a further embodiment the present invention includes an ophthalmic composition wherein the concentration of olopatadine hydrochloride is 0.01% w/v to 0.15% w/v.

In a further embodiment the present invention includes an ophthalmic composition wherein the concentration of olopatadine hydrochloride is 0.1% w/v to 0.1% w/v.

In a further embodiment the present invention includes an ophthalmic composition wherein the concentration of ketorolac tromethamine is 0.1% w/v to 0.5% w/v.

In a further embodiment the present invention includes an ophthalmic composition wherein the concentration of ketorolac tromethamine is 0.1% w/v-0.4% w/v.

In a further embodiment the present invention includes an ophthalmic composition wherein the concentration of olopatadine hydrochloride is 0.1% w/v.

In a further embodiment the present invention includes an ophthalmic composition wherein the concentration of ketorolac tromethamine is 0.4% w/v.

In a further embodiment the present invention includes an ophthalmic composition comprising benzalkonium chloride.

In a further embodiment the present invention includes an ophthalmic composition, wherein the concentration of benzalkonium chloride is 0.005% w/v.

In a further embodiment the present invention includes an ophthalmic composition, wherein the composition is provided as a kit containing a 1-10 ml plastic dropper designed for topical administration of the composition.

In a further embodiment the present invention includes an ophthalmic composition wherein the composition is provided as a kit containing a 5 ml plastic dropper designed for topical administration of the composition.

In a further embodiment the present invention includes an ophthalmic composition wherein the composition may be used for treatment of allergic conjunctivitis by applying 1-4 drops of the composition to each eye per day.

In a further embodiment the present invention includes an ophthalmic composition wherein the composition may be used for treatment of ocular surface allergy, by applying 1-4 drops of the composition to each eye per day.

### BRIEF DESCRIPTION OF THE FIGURES

FIG. 1: Graphical representation of itching in study group and the comparator group.

FIG. 2: Graphical representation of cilliary hyperemia in study group and the comparator group.

FIG. 3: Graphical representation of conjunctival hyperemia in study group and the comparator group.

FIG. 4: Graphical representation of episcleral hyperemia in study group and the comparator group.

FIG. 5: Graphical representation of chemosis in study group and the comparator group.

FIG. 6: Graphical representation of patients with mucous discharge in study group and the comparator group.

### DETAILED DESCRIPTION OF THE INVENTION

Seasonal allergic conjunctivitis is the most common allergic disease to affect the eye, typically drawn by airborne allergens such as pollen, grass, weeds and animal dander. It is a type 1 hypersensitivity reaction mediated by IgE in response to these environmental antigens. The principal symptom of seasonal allergic conjunctivitis is ocular itching, other symptoms include: conjunctival hyperemia, tearing, mucus discharge, chemosis and lid edema. (Yayli V, Demirlenk I, Tatlipinar S, Ozbay D, Esme A, Yildirim C et al. “Comparative study of 0.1% Olopatadine Hydrochloride and 0.5% Ketorolac Tromethamine in the Treatment of Seasonal Allergic Conjunctivitis” Acta Ophthalmol. Scand. 2003, 81, 378-382).

The traditional treatment of allergic conjunctivitis (ocular surface allergy) consists of a reduction in exposure to allergens by limiting outdoor activities during times of high pollen or ragweed counts and avoidance of animal dander and dust. The use of eyewashes, irrigating solutions, and tear substitutes flushes the antigen from the eye and provides temporary alleviation of the symptoms. However, the identification of the role of mast cell degranulation, histamine release and the production of arachidonic acid metabolites in the pathogenesis of allergic conjunctivitis has resulted in a pharmacological basis of treatment. (Raizman M B, “Results of a Survey of Patients with Ocular Allergy Treated with Topical Ketorolac Tromethamine”. Clinical Therapeutics. 1995, 17(5), 882-890).

The combination of olopatadine with ketorolac in a single composition in the concentrations stated herein is novel.

### Clinical Aspects

A randomized, multi-centric geographically, open label study to compare the safety and efficacy of the combination of olopatadine hydrochloride 0.1% and ketorolac tromethamine 0.4% w/v ophthalmic solution with olopatadine hydrochloride 0.1% w/v used twice daily for three weeks in adult patients with seasonal allergic conjunctivitis, was completed. The primary objective of this randomized, multi-centric geographically diverse study was to compare the safety and efficacy of the combination of olopatadine and ketorolac with olopatadine in seasonal allergic conjunctivitis.

A combination of olopatadine hydrochloride 0.1% w/v and ketorolac tromethamine 0.4% w/v was compared with olopatadine hydrochloride 0.1% w/v alone in the treatment of patients with seasonal allergic conjunctivitis. The test drug was the combination of olopatadine hydrochloride with ketorolac tromethamine compared to olopatadine hydrochloride 0.1% w/v, as three commercially available brands on the market: Winolap®, Opat® and Olo®. These three commercially available brands, as the comparator drug, were provided to the investigators. Any of the three brands of the comparator drug were prescribed by the investigator to the patients per their choice of availability and cost.

The study showed that the combination (FDC) of olopatadine hydrochloride 0.1% w/v with ketorolac tromethamine 0.4% w/v (the combination of olopatadine and ketorolac) is more effective in relieving the signs and symptoms of seasonal allergic conjunctivitis than olopatadine hydrochloride 0.1% w/v and was similar in safety.

The patients administered one drop twice daily in each eye for twenty-one days during three weeks. The patients received study medications as well as comparator drug for the entire period. There were five scheduled visits, with screening periods of thirty days and a treatment period of twenty-one days as per protocol. At the end of each visit physicians also rated tolerance and the patient rated acceptability.

The efficacy parameters like itching, hyperemia (cilliary, conjunctival, episcleral), chemosis and mucus discharge were compared between groups and scored. The efficacy of the medications was evaluated by comparing the reduction in efficacy parameters, signs and symptoms of allergic conjunctivitis between the treatment groups, based on the twenty-one day period.

The efficacy of the combination of olopatadine-ketorolac was compared with the efficacy of olopatadine in reducing the symptoms of a seasonal allergic conjunctivitis like itching, hyperemia, chemosis, and ocular mucous discharge.

The tolerability and acceptability of the combination of olopatadine-ketorolac was compared with the tolerability and acceptability of olopatadine in patients with seasonal allergic conjunctivitis.

The efficacy of the study medications was evaluated by comparing the reduction in signs and symptoms of allergic conjunctivitis between the treatment groups. Reduction in the signs and symptoms of allergic conjunctivitis was based on evaluation of efficacy parameters till the end of twenty-one days.

For the improvement of allergic conjunctivitis, symptoms like itching, hyperemia, chemosis and mucous discharge were used. The symptoms of allergic conjunctivitis like itching, hyperemia and chemosis were scored as 0, 1, 2, 3 and 4 and mucous discharge was graded as absent or present.

| Parameter | Finding | Score |
|---|---|---|
| Itching | Absent | 0 |
| Hyperemia | Mild | 1 |
| Chemosis | Moderate | 2 |
| | Severe | 3 |
| | Extremely severe | 4 |
| Mucous discharge | Absent | a |
| | Present | b |

Interpretation: •Minimum score: 0; •Maximum score: 4; the higher the score the more severe will be the symptoms of allergic conjunctivitis. In the report, each symptom is graded based on the severity.

At the end of the each visit physicians rated tolerance as Excellent (No adverse drug reactions), Good (Mild adverse reaction not requiring other treatment or necessitating drug withdrawal), Fair (Adverse reactions requiring treatment without hospitalization or necessitating drug withdrawal and Poor (Severe or serious reaction requiring hospitalization and withdrawal of drug) and the patient rated acceptability as Good (No untoward incidence), Fair (Mild adverse reaction not interfering day to day activities) and Poor (Adverse reactions affecting regular day to day activities).

The safety of the combination of olopatadine-ketorolac was compared with olopatadin. The safety assessment parameters included the number of patients having adverse events, the types of adverse events, and the number of unexpected, severe or serious adverse events in the two arms.

The study showed that the combination of olopatadine hydrochloride 0.1% with ketorolac tromethamine 0.4% (olopatadine-ketorolac) was more effective in relieving the signs and symptoms of seasonal allergic conjunctivitis than olopatadine hydrochloride 0.1% and was similar in safety.

Efficacy evaluation has been done for first 100 completed cases. The efficacy parameters like itching, hyperemia (cilliary, conjunctival, episcleral), chemosis and mucus discharge were compared between groups.

Itching:

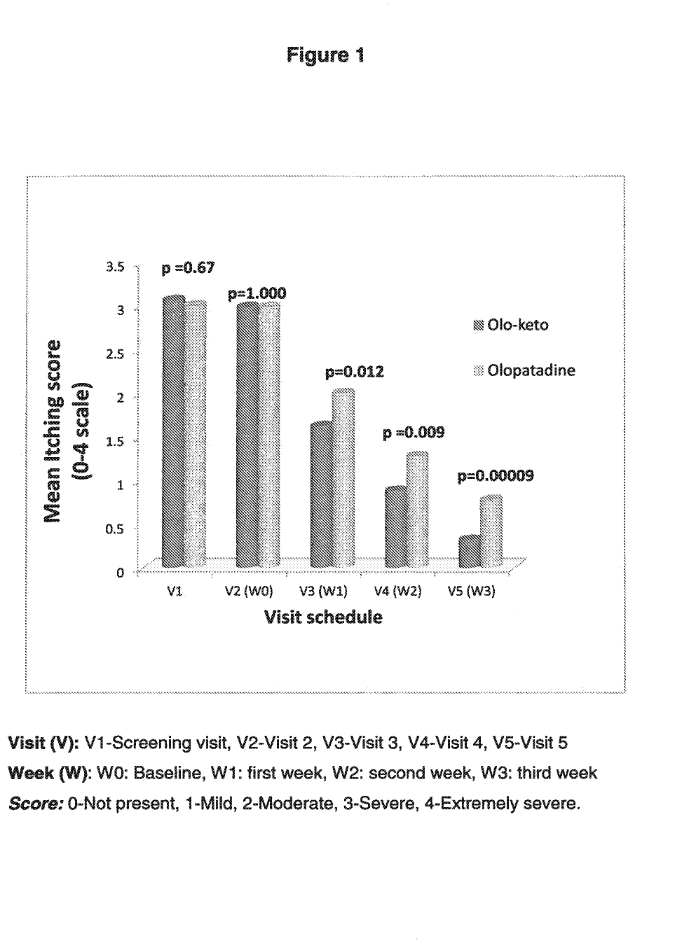

At the baseline, all the patients enrolled in each group had a similar number of complaints in itching varying from mild to extremely severe in severity with a mean itching score of 3.06±0.91 in the combination of olopatadine and ketorolac group and 3±0.67 in the olopatadine group. There was a significant (p<0.05) decrease in the itching during visit 3 (1.62±0.85), visit 4 (0.88±0.82) and visit 5 (0.32±0.55) in the combination of olopatadine and ketorolac group when compared to visit 3 (2±0.60), visit 4 (1.28±0.67) and visit 5 (0.78±0.58) of olopatadine group (FIG. 1).

This study showed that the FDC of the combination of olopatadine and ketorolac is more effective than olopatadine in relieving mild to extremely severe itching which is a hallmark of allergic conjunctivitis. The combination of olopatadine and ketorolac showed a significant decrease in itching during first (p=0.012), second (p=0.009) and third (p=0.00009) week of treatment when compared to olopatadine. Both the treatment groups had similar baseline score (p=1.000). The significant reduction observed in the combination of olopatadine and ketorolac group in itching might be because of the synergistic action of olopatadine and ketorolac. Olopatadine with the dual action of mast cell stabilization and antihistaminic activity inactivates the immediate cascade of histamine-associated responses during the early phase of the hypersensitivity reaction and also the more chronic or later phase of the disease by mast cell stabilization. However, it shows no effect on the newly formed mediators such as prostaglandins, (except PGD₂) which continue to produce ocular allergy symptoms for several hours after allergen exposure. The addition of ketorolac helps in controlling the newly formed mediators.

The combination of histamine and prostaglandin are known to be additive in producing ocular itching. The addition of ketorolac to olopatadine helps in inhibiting the prostaglandin synthesis, in addition to mast cell stabilization and antihistaminic activity of olopatadine. Ketorolac specifically reduces PGD2, PGE₂ and PGI₂ which are known to be potent itch-producing substances in the conjunctival tissue.

Hyperemia (Cilliary):

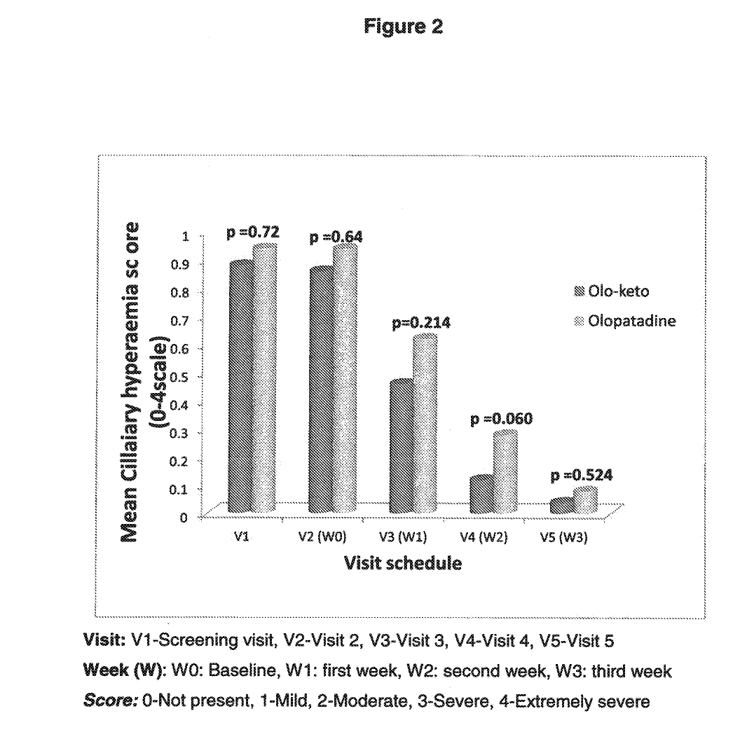

At the baseline, all the patients enrolled in each group had similar number of complaints in cilliary hyperemia varying from mild to severe in severity with a mean cilliary hyperemia score of 0.88±0.79 in the group and 0.94±0.91 in the olopatadine group. During visit 2 and visit 3, the mean cilliary hyperemia score was 0.86±0.80 and 0.46±0.61 in the combination of olopatadine and ketorolac group and 0.94±0.91 and 0.62±0.66 in the olopatadine group. However, the combination of olopatadine and ketorolac significantly reduced (p=0.06, p=0.52) cilliary hyperemia during visit 4 (0.12±0.32), and visit 5 (0.04±0.28) when compared to visit 4 (0.28±0.49) and visit 5 (0.08±0.34) of the olopatadine group (FIG. 2).

Hyperemia (Conjunctival):

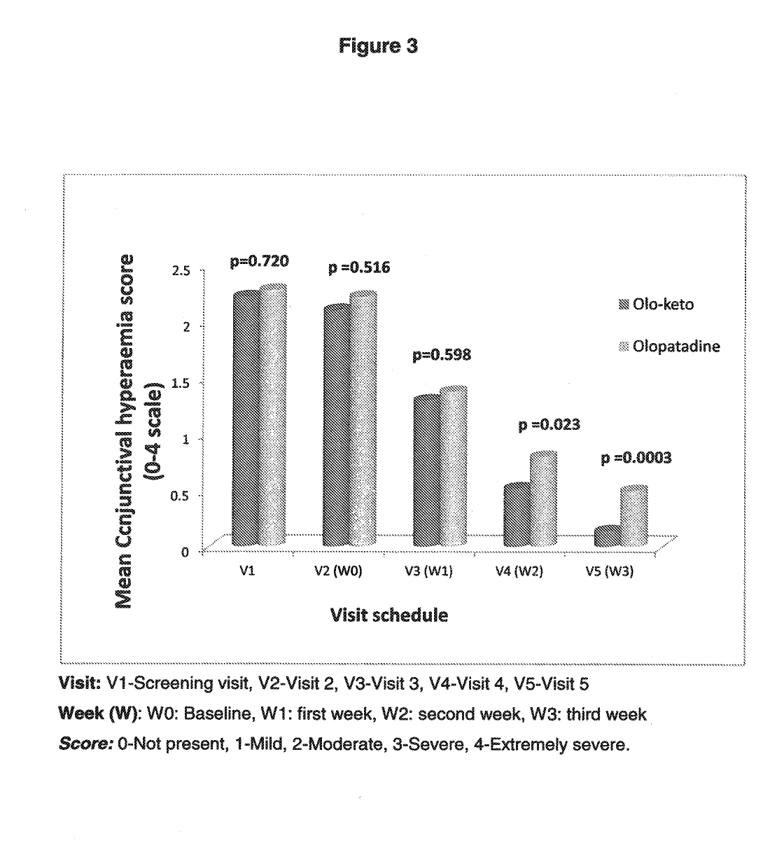

During baseline, the number of patients with conjunctival hyperemia varied from mild to extremely severe in severity and was similar in both the treatment groups. The mean conjunctival hyperemia score was 2.22±0.84 in the combination of olopatadine and ketorolac group and 2.28±0.83 in Olopatadine group. Both the treatment showed decrease in the Conjunctival hyperemia during visit 2; The combination of olopatadine and ketorolac 2.1±0.09, olopatadine 2.2±0.84 and visit 3; The combination of olopatadine and ketorolac 1.3±0.83, olopatadine 1.38±0.86; no statistically significant difference was observed between the two treatments. However, during visit 4 and 5 the combination of olopatadine and ketorolac (0.52±0.61; 0.14±0.4) significantly reduced (p=0.02, p=0.0003) conjunctival hyperaemia when compared to olopatadine (0.8±0.60; 0.5±0.54) (FIG. 3).

Hyperemia (Episcleral):

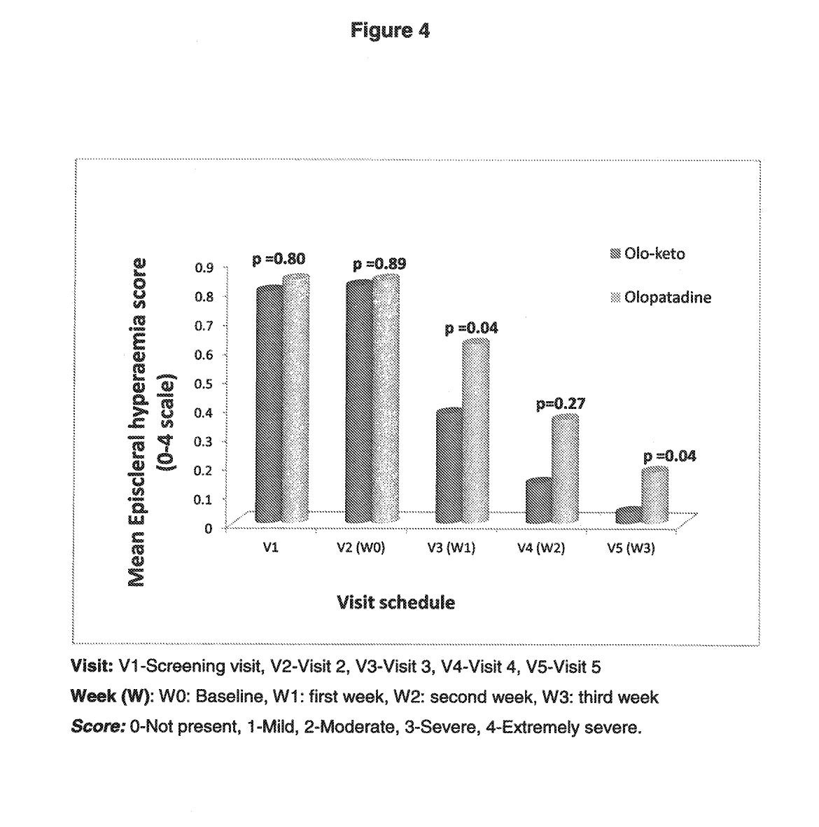

During baseline, the mean episcleral hyperemia score was 0.8±0.80 in the combination of olopatadine and ketorolac group and 0.84±0.79 in the olopatadine group. The combination of olopatadine and ketorolac group (0.38±0.53) showed a significant decrease (p=0.43) in episcleral hyperemia during visit 3 when compared to visit 3 (0.62±0.63) of the olopatadine group. However, no significant difference was observed between visit 2 (0.82±0.774), visit 4 (0.14±0.40) and visit 5 (0.04±0.28) of the combination of olopatadine and ketorolac group and visit 2 (0.84±0.79), visit 4 (0.36±0.56) and visit 5 (0.18±0.38) of the olopatadine group (FIG. 4).

The combination of olopatadine and ketorolac was found to be more effective than olopatadine in reducing the conjunctival hyperemia. The combination of olopatadine and ketorolac showed a significant decrease in conjunctival hyperemia during second (p=0.02) and third (p=0.0003) week of treatment and showed greater reduction on first week, although the values were not statistically significant when compared to olopatadine.

The combination of olopatadine and ketorolac showed a significant decrease in episcleral hyperemia during first (p=0.04) and third (p=0.04) week of treatment when compared to olopatadine. The combination of olopatadine and ketorolac showed a greater reduction in cilliary hyperaemia when compared to olopatadine on all scheduled visit, although the values were not statistically significant. The significant decrease in conjunctival hyperaemia could be possibly due to the combined effect of olopatadine and ketorolac. Olopatadine offers the advantage of rapid relief of symptoms by immediate antihistaminic activity coupled with long term benefits of mast cell stabilization whereas ketorolac inhibits the synthesis of prostaglandins (PGE₂ and PGI₂) which are highly hyperemic and helps in the relief of conjunctival hyperemia.

Chemosis:

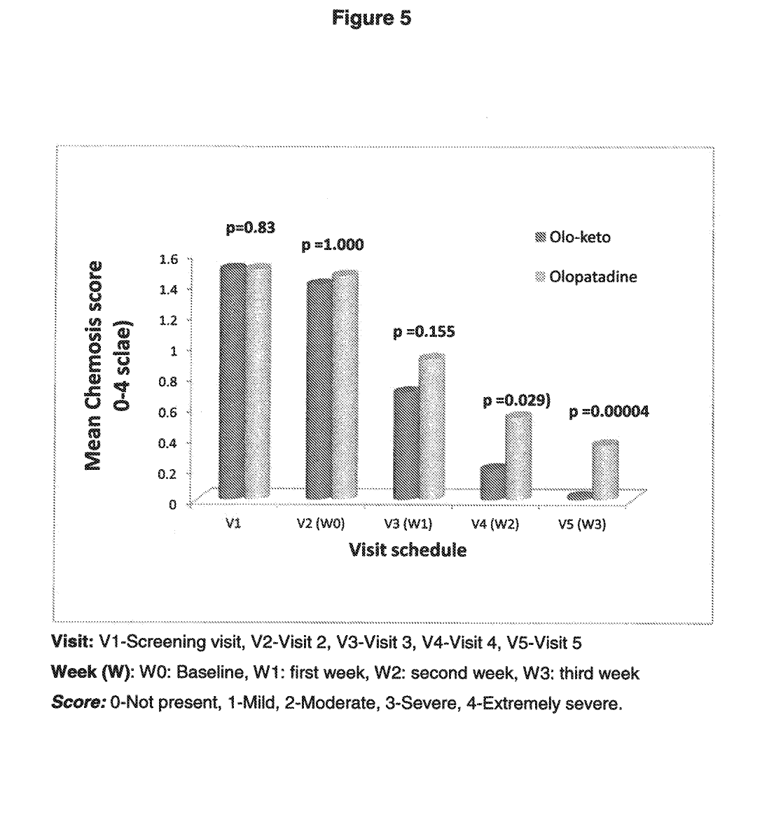

During baseline, the number of patients with chemosis varying from mild to extremely severe in severity was similar in both the treatment groups with mean chemosis score of 1.5±0.95 in the combination of olopatadine and ketorolac group and 1.5±0.99 in olopatadine group. Both the treatment groups showed decrease in the chemosis during visit 2; the combination of olopatadine and ketorolac 1.4±0.97, olopatadine 1.46±0.95 and visit 3; the combination of olopatadine and ketorolac 0.7±0.67, olopatadine 0.92±0.72; no statistically significant difference in reducing chemosis was observed between the two treatments. However, during visit 4 and 5 the combination of olopatadine and ketorolac (0.2±0.46; 0.54±0.57) significantly reduced (p=0.029, p=0.00004) conjunctival hyperemia when compared to olopatadine (0.54±0.57; 0.3±0.52) (FIG. 5).

The combination of olopatadine and ketorolac also showed a significant reduction in chemosis during second (p=0.02) and third (p=0.00004) week when compared to olopatadine. There was no difference in the baseline (p=1.00) scores and both the treatment groups showed decrease in chemosis during the first (p=0.15) week. In a study conducted by Abelson M B et al. Olopatadine showed significant decrease (p=0.03) in chemosis during fourth week of treatment when compared to placebo. However, in our study the combination of olopatadine and ketorolac showed a significant decrease in chemosis during second (p=0.029) and third (0.0004) week of treatment when compared to olopatadine hydrochloride.

Mucous Discharge:

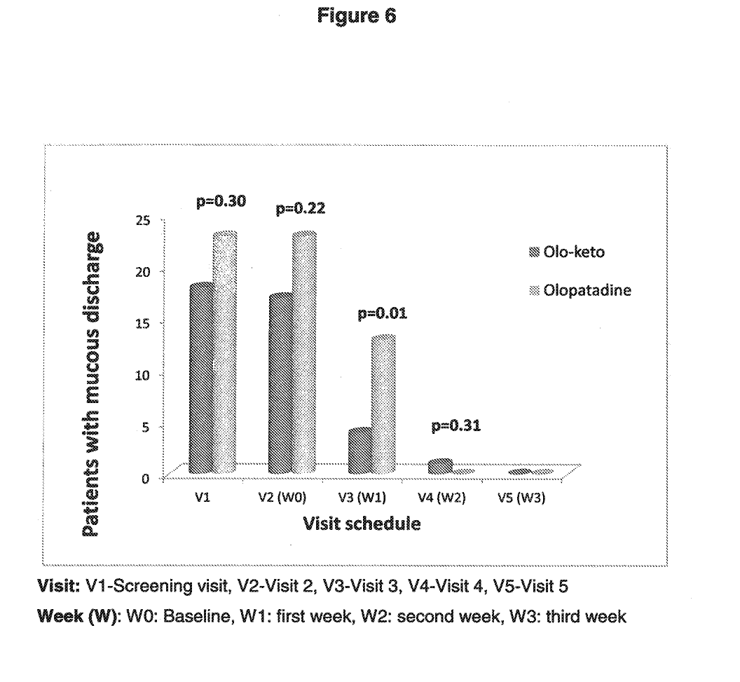

Both the treatment groups were equally effective in reducing the complaints of mucus discharge by the end of the study. However, the combination of olopatadine and ketorolac significantly reduced (p<0.01) mucus discharge during visit 3 when compared to olopatadine. In the final scheduled visit all the enrolled patients were free from the complaints of mucus discharge. FIG. 6 shows the number of patients with the complaints of mucus discharge in each group.

The combination of olopatadine and ketorolac showed a statistically significant reduction (p=0.016) in mucous discharge during first week when compared to olopatadine. After first week of treatment, both the treatment groups showed similar reduction in mucus discharge.

The significant reduction observed in olopatadine-ketorolac group in itching might be because of the synergistic action of olopatadine and ketorolac. Olopatadine with the dual action of mast cell stabilization and antihistaminic activity inactivates the immediate cascade of histamine-associated responses during the early phase of the hypersensitivity reaction and also the more chronic or later phase of the disease by mast cell stabilization. The addition of ketorolac helps in controlling the newly formed mediators.

The combination of olopatadine with ketorolac showed excellent tolerability when compared to olopatadine as per investigators' assessment and good acceptability when compared to olopatadine as per patients' assessment.

The combination of olopatadine and ketorolac was found to be more effective than olopatadine in reducing the conjunctival hyperemia. The significant decrease in conjunctival hyperemia could be due to the combined effect of olopatadine and ketorolac. Olopatadine offers the advantage of rapid relief of symptoms by immediate antihistaminic activity coupled with long term benefits of mast cell stabilization whereas ketorolac inhibits the synthesis of prostaglandins (PGE₂ and PGI₂) which are highly hyperemic therefore helping in the relief of conjunctival hyperemia. (Raizman M B, “Results of a Survey of Patients with Ocular Allergy Treated with Topical Ketorolac Tromethamine”. Clinical Therapeutics. 1995, 17(5), 882-890; Woodward D F, Nieves A L, Hawley S B, Joseph R, Merlino G F, Spada C S. “The Pruitogenic and Inflammatory Effects of Prostanoid in the Conjunctiva” Journal of Ocular Pharmacology and Therapeutics. 1995, 11(3), 339-347).

The combination of olopatadine and ketorolac showed a significant decrease in episcleral hyperaemia during first and third week of treatment when compared to olopatadine.

The combination of olopatadine and ketorolac also showed a significant reduction in chemosis during the second and the third week when compared to olopatadine. In a study conducted by Abelson M B et al. olopatadine showed significant decrease in chemosis during fourth week of treatment when compared to placebo. (Abelson M B, Turner D. “A Randomization, Double Blind, Parallel Group Comparison of Olopatadine 0.1% Solution Versus Placebo for Controlling the Signs and Symptoms of Seasonal Allergic Conjunctivitis and Rhinoconjunctivitis”. Clinical Therapeutics. 2003: 931-947) However, in this study the combination of olopatadine with ketorolac showed a significant decrease in chemosis during the second and the third week of treatment when compared to olopatadine hydrochloride.

Olopatadine-ketorolac showed a statistically significant reduction in mucous discharge during first week when compared to olopatadine. After the first week of treatment, both the treatment groups showed similar reduction in mucus discharge.

The combination of olopatadine and ketorolac was similar in safety to olopatadine with three patients in each group experiencing an adverse event which were mild transient in nature and no treatment was required to treat adverse events. The adverse event was observed immediately after the installation of eye drop.

The combination of olopatadine and ketorolac showed excellent tolerability when compared to olopatadine as per investigators assessment and good acceptability when compared to olopatadine as per patient's assessment. The combination of olopatadine and ketorolac was found to be more effective in reducing the signs and symptoms of seasonal allergic conjunctivitis and was similar to olopatadine in safety. The FDC was found to be more acceptable and tolerable when compared to olopatadine.

What is claimed is:

1. A topical ophthalmic composition for treating or preventing seasonal allergic conjunctivitis in a human patient, wherein the ophthalmic composition comprises a combination of a non-steroidal anti-inflammatory drug with an antihistamine intended for an ophthalmic use.

2. The composition according to claim 1, wherein the non-steroidal anti-inflammatory drug is selected from: indomethacin, diclofenac, flurbiprofen, ketorolac, suprofen, bromfenac and nepafenac.

3. The composition according to claim 1, wherein the antihistamine is selected from: olopatadine, ketotifen, epinastine, alcaftadine, azelastine, cetitizine, axelastine, emedastine, bepotasine, chlorpheneramine, clemastine, diphenhydramine, loratadine and pheniramine.

4. The ophthalmic composition, according to claim 1, comprising a combination of olopatadine hydrochloride and ketorolac tromethamine.

5. The ophthalmic composition, according to claim 4, wherein the concentration of olopatadine hydrochloride is 0.01% w/v to 0.15% w/v.

6. The ophthalmic composition, according to claim 4, wherein the concentration of ketorolac tromethamine is 0.1% w/v to 0.5% w/v.

7. The ophthalmic composition, according to claim 5, wherein the concentration of olopatadine hydrochloride is 0.1% w/v to 0.1% w/v.

8. The ophthalmic composition, according to claim 6, wherein the concentration of ketorolac tromethamine is 0.1% w/v to 0.4% w/v.

9. The ophthalmic composition, according to claim 1, comprising a combination of olopatadine hydrochloride 0.1% w/v and ketorolac tromethamine 0.4% w/v.

10. The ophthalmic composition, according to claim 9, further comprising a preservative.

11. The composition according to claim 10, wherein the preservative is selected from: chlorine dioxide, polixetonium, polyhexamethylene biquanide, polyquad and sodium perborate.

12. The ophthalmic composition, according to claim 9, further comprising benzalkonium chloride.

13. The ophthalmic composition, according to claim 12, wherein benzalkonium chloride is present in the amount of 0.005% w/v.

14. The ophthalmic composition of claim 13, wherein the composition is provided as a kit containing a 5 ml plastic dropper designed for topical administration of the composition.

15. The ophthalmic composition of claim 13, wherein the composition may be used for treatment of seasonal allergic conjunctivitis by applying 1-4 drops of the composition to each eye per day.
